(19) United States
(12) Patent Application Publication
(10) Pub. No.: US 2007/0083261 A1
(43) Pub. Date: Apr. 12, 2007

(54) Method of maintaining the preoperative dimensions of the eye in an intraocular lens placement procedure

(76) Inventor: David Michael Colvard, Pacific Palisades, CA (US)

Correspondence Address: KOPPEL, PATRICK & HEYBL, 555 ST. CHARLES DRIVE, SUITE 107, THOUSAND OAKS, CA, 91360, US

(21) Appl. No.: 11/443,667

(22) Filed: May 30, 2006

Related U.S. Application Data
(63) Continuation-in-part of application No. 11/378,606, filed on Mar. 17, 2006, now abandoned.
Application No. 11/378,606 is a continuation-in-part of application No. 11/245,904, filed on Oct. 7, 2005.

(51) Int. Cl. A61F 2/16; A61F 9/013

(52) U.S. Cl. 623/6.39; 623/907

(57) ABSTRACT

The spatial relationship of structures within the eye, and more particularly the distance from a surface of the cornea to the posterior of the crystalline lens, or the posterior of the lens capsule and the retina or the cornea and the retina, is determined preoperatively, thus establishing the preoperative anatomical relationships. The distance relationship is reestablished or a new preferred distance relationship is established interoperatively using the same or similar techniques. One or more spacing means are implanted in the lens capsule of a patient at a position adjacent to an implanted posterior chamber intraocular lens following removal of the natural crystalline lens. The one or more spacing means in combination with the intraocular lens produce a desired spatial relationship for the lens capsule which may approximate the spatial relationship of the lens capsule measured prior to removal of said crystalline lens or may be a different preferred relationship. The combination of the thicknesses of the IOL and spacing means allows the originally determined relationships to be substantially duplicated or modified.

This is a continuation-in-part of U.S. patent application Ser. No. 11/378,606 filed Mar. 17, 2006 which is a Continuation-In-Part of U.S. patent application Ser. No. 11/245,904 filed Oct. 7, 2005.

The invention is directed to methods for measuring the dimensions and spacing within a human eye so that, as part of an ophthalmic surgical procedure, for example a lens removal and IOL implantation in a cataract extraction procedure, or other ophthalmic surgical procedures, the spacing in the eye and the spatial relationships between various structures in the eye immediately postoperatively can be returned to substantially the same relationship as preoperatively or a desired different spatial relationship. For example, in an IOL placement procedure, one or more spacers can be placed anterior to an intraocular lens (IOL) to maintain the dimensions of the eye existing prior to the cataract removal procedure. This includes controlling the dimensions of the anterior and/or posterior chamberln the instance of an IOL procedure, the measurement procedure, and the subsequent implantation of an artificial lens and space fillers are beneficial in providing correct vision but may be particularly beneficial when the patient is a high myope.

### BACKGROUND

The human eye comprises a spherical structure that includes a cornea, which comprises the outer surface of the eye, a crystalline lens centrally located in a lens capsule behind a pupil and retina, optic and other nerves on the rear wall of the eye. These nerves connect the eyes to the brain, and particular areas of the brain that are in neural communication with the eyes. Images pass through the cornea and a pupil, which is centrally located in the iris, and are focused by the lens onto the image receptors at the rear of the eye.

Each eye forms an image upon a vast array of light sensitive photoreceptors of the retina. The outer cover of the eye, or cornea, protects the lens and acts as a colorless filter to refract light onto the iris and pupil. The iris corresponds to the aperture in a camera and contains muscles that alter the size of the pupil to control the amount of light that enters the eye. The natural crystalline lens located posterior to the pupil has a variable shape under the indirect control of peripheral ciliary muscles. Having a refractive index higher than the surrounding media, the crystalline lens gives the eye a variable focal length, allowing accommodation to objects at varying distances from the eye. Much of the remainder of the eye is filled with fluids under pressure that help the eye maintain its shape.

The human eye is susceptible to numerous disorders, diseases and optical deficiencies. Corrective glasses, contact lenses or laser sculpting typically addresses optical deficiencies. Besides optical deficiencies, several diseases that can affect the natural crystalline lens or the optical nerve or macula can degrade vision. For example, cataracts interfere with vision by causing a cloudy or opaque discoloration of the natural lens of the eye. Cataracts often result in partial or complete blindness. If this is the case, the crystalline lens can be removed and replaced with an intraocular lens (IOL). As addressed below, cataract lens removal presents addition optical problems and may result in retinal detachment and macular degeneration. This is specifically true for a myopic patient, and in particular one who is highly myopic.

Intraocular lenses (IOLs) have proven to be very successful in restoring normal vision to individuals following removal of a natural crystalline lens clouded by the presence of a cataract. The normal human natural lens is thicker in its center than an IOL and this may present a problem with any patient. In particular, in an individual considered to be a high myope (requiring an optical correction greater than about 5 diopters) the natural lens may have a thickness as great as 5 mm. Following implantation of an IOL the posterior lens capsule wall tends to shrink and wrap around the IOL. Because of the greater thickness of the removed lens, the shrinking capsule can result in the loss of several millimeters of capsule depth, and the remaining posterior capsule wall and vitreous fluid shifts forward. This forward movement of the vitreous fluid can cause a retinal detachment and initiate macular degeneration.

This situation was recognized by Giovinazzo in U.S. Pat. No. 4,710,195 wherein he states:
“patients with high myopia are recommended by many to have an implant lens not for optical correction but to prevent the mobility of the posterior capsule. This mobility and subsequent anterior-posterior movement of the vitreous removes many of the benefits of extra capsular surgery.”

However, IOLs have not been found to adequately prevent the forward movement of the rear wall of the capsule.

This problem may be of an even greater concern should the posterior membrane tear or have to be later removed or opened due to posterior capsular opacification (PCO), which is normal in about 30 % of IOL placements. PCO can occur due to the proliferation of epithelial cells at the periphery of the posterior capsule wall that can grow and spread under the IOL on the inside surface of the wall and cause opacification. The normal IOL structure may not be adequate to prevent this PCO.

Lenses have been designed with a ring as part of the rear surface of the IOL in an attempt to prevent opacification and forward movement of the vitreous. U.S. Pat. No. 4,244,060 to Hoffer shows a plano-convex posterior chamber lens with a rearward projecting, substantially annular ridge or lip which presses against the rear capsule. The lip is stated to limit “the progress of vitreous humor toward the anterior chamber after a decision, and may limit lens fiber growth on the posterior capsule within the lip region.” Other lens designs are intended to prevent the growth of cells onto the IOL, and thus inhibit posterior capsule opacification, by providing a peripheral wall having an outer corner edge with a sharp outer corner resting against the capsule wall to substantially retard or prevent the growth of cells onto the lens side walls and-eventually extending across the rear surface of the IOL.

While not specifically designed for high myopes, another approach is to provide an optic which totally fills the posterior capsules. One approach is shown by Siepser, U.S. Pat. No. 4,556,998 and U.S. Pat. No. 5,147,394, which show an expandable hydrogel. A lens of about 2 to 5 mm in diameter and an appropriate thickness is formed from a dry hydrogel. That lens is then implanted in the posterior capsule where the natural fluids wet the hydrogel which swells to a diameter of 6-14 mm along with an increase in thickness which may fill the depth of the capsule.

A still further alternative is to provide an inflatable lens such as shown in U.S. Pat. No. 4,619,662 to Juergens or U.S. Pat. No. 4,822,360 to Deacon. In these designs, an inflatable, transparent sac or bag is placed in the posterior capsule. The bag is inflated to its intended dimension by filling with a fluid, which may be a polymerizable elastomer, to create an optically correct, transparent lens with proper vision correction. These lenses can be made to fill the posterior capsule.

Attempts have also been made to provide spatial lens designs to meet the optical requirements of high myopes. These include the use of thicker and greater diameter optics, or lenses with a much greater rearward angulation. U.S. Pat. No. 3,866,249 to Flom shows a thick biconvex IOL which is said to provide support for the hyloid membrane and the vitreous humor.

A further alternative which may be used to provide large optical corrections is to implant two lenses in a single eye. The lenses may be separated from each other by a spacer, or a ring shaped frame may be provided with a central circular opening to receive a lens of desired optical characteristics. This lens insert could also be very thick to provide telescopic properties. U.S. Pat. No. 5,769,890 to McDonald is directed to placement of a second IOL, preferably behind the iris but in front of the capsule containing a first IOL, to correct optical errors resulting from the selection of a first, prior implanted IOL. U.S. Pat. No. 6,616,692 and U.S. Pat. No. 6,797,004 to Brady and Glick also show implantation of two IOLs, both providing optical correction. The '004 patent shows a peripheral holder with the lens centrally located therein or two optical lenses separated by an intermediate solid spacer to maintain a preset space between the lenses.

Other examples of peripheral rings to hold an IOL are shown in U.S. Pat. No. 5,628,798 and Published application 2002/0128710 Eggleston, et al. U.S. Pat. No. 6,007,579 to Lipshitz et al shows a telescopic optic held in a circular ring. U.S. Pat. No. 5,876,442 is a further example of a telescopic optic between two spaced apart carrier rings. Other examples of the use of a peripheral ring to hold an IOL are U.S. Pat. No. 5,824,074 to Koch and U.S. Pat. No. 5,628,795 and RE 34,998 to Langerman. These rings are positioned radially outward from the optic and are used to hold the optic and maintain the diameter of the capsule or provide accommodation and are not intended to, and do not finction, to space the optic rearwardly to maintain the position of the posterior capsule wall and the volume of the vitreous chamber.

Also, separate rings have been suggested to maintain the normal capsule diameter. U.S. Pat. No. 5,843,184 shows an example of a tension ring placed in the capsule solely for the purpose of maintaining the diameter of the capsule and does not hold or space the optic and will not maintain the capsule volume and vitreous space. Dick discloses the use of a closed, folded, rigid capsular ring inserted prior to IOL Placement to maintain a fixed capsule diameter (Dick, H.B., “Closed Foldable Capsular Rings”, J Cataract Refract. Surg., 31, pp 467-471 (March 2005).

However, none of these devices have proven to be suitable to maintain capsule depth and vitreous chamber volume and depth unchanged. There is therefore a need for a suitable means for maintaining the shape and volume of the posterior capsule containing an IOL and the position of the rear wall of the capsule in relationship to the rear of the eye, so the vitreous will not move forward, in turn helping the macula and retina to remain intact. This would be useful in all cataract surgery and in particularly in aphakic myopics.

### SUMMARY

The spatial relationship of structures within the eye, such as the distance from the a surface of the cornea to a posterior surface of the crystalline lens capsule and from the cornea or the posterior surface of the lens capsule to the retina is measured preoperatively, for example by using ultrasound, partial coherence interferometry, optical coherence tomography or laser measuring techniques or by any other means known to the art, thus establishing the preoperative anatomical relationships. A surgical procedure, such as an IOL implantation is performed and spacing means are provided to restore those premeasured spatial relationships or a predetermined new spacing. The spacing means can include, for example spacers, rings, inflatable structures or thick or multiple lenses. These spacing means aid in maintaining the normal depth of the patient's anterior and posterior capsule and prevent forward movement of the vitreous and retinal detachment that may occur as a result of such movement. The distance relationship is reestablished intraoperatively using the same or similar measuring techniques, the combination of the thicknesses of the IOL and spacing means providing the means to substantially duplicate or modify the spacing measured preoperatively.

### BRIEF DESCRIPTION OF DRAWINGS

FIG. 1 is a schematic cutaway side view of the human eye.

FIG. 2 is a schematic cutaway side view showing an IOL located in the posterior capsule of a human eye.

FIG. 3 is a schematic cutaway side view of an IOL placed in the posterior capsule along with spacers incorporating features of the invention positioned anterior to the IOL.

FIG. 4 is a schematic cutaway side view of an IOL placed in the posterior capsule along with second arrangement of multiple spacers positioned in the capsule anterior to the IOL.

FIG. 5 is a side perspective view of a disc incorporating features of the invention.

FIG. 6 is a side perspective view of a first embodiment ring incorporating features of the invention.

FIG. 7 is a side view of a modification of FIG. 6 showing a ring with angled lower surface cutaway along line 7-7 of FIG. 6 second embodiment of a ring.

FIG. 8 is a cross sectional view of a combination of an IOL, angled ring and disks as it would appear in a posterior capsule.

FIG. 9 is a schematic cutaway view of a further embodiment showing an IOL plus multiple spacers.

FIG. 10 is a cross sectional view of a further embodiment of an 1 10L with a ring and spacer.

### DETAILED DESCRIPTION

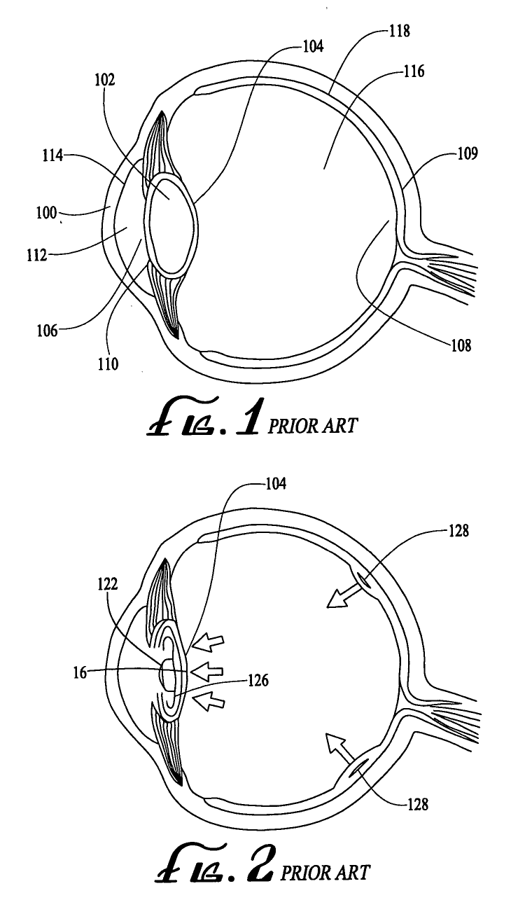

The structure of the human eye is shown schematically in FIG. 1. The human eye comprises a spherical structure which includes the cornea 100, which comprises the outer surface of the eye, a crystalline lens 102 centrally located in a lens capsule 104 behind the pupil 106 and the retina, optic and other nerves 108 on the rear wall of the eye that connect the eyes to the brain, and particular areas of the brain that are in neural communication with the eyes. Images pass through the cornea 100 and iris 110, which is centrally located in the pupil 106, and are focused by the lens 102 onto the image receptors at the rear of the eye.

Each eye forms an image upon a vast array of light sensitive photoreceptors of the retina. The outer cover of the eye, or cornea, protects the lens and acts as a colorless filter to refract light onto the iris and pupil. The iris 110 corresponds to the aperture in a camera and contains muscles that alter the size of the pupil to control the amount of light that enters the eye. The natural crystalline lens 102 located just behind or posterior to the pupil 106 has a variable shape under the indirect control of the peripheral ciliary muscles. Having a refractive index higher than the surrounding media, the crystalline lens gives the eye a variable focal length, allowing accommodation to objects at varying distances from the eye.

Much of the remainder of the eye is filled with fluids under pressure that help the eye maintain its shape. For example, the aqueous humor (fluid) 112 fills the anterior chamber 114 between the cornea 100 and the pupil 106, and the vitreous humor (gel) 116 fills the majority of the volume of the eye in the vitreous chamber 118 that is located between the lens 102 and the retina and other optic nerves 108.

The human eye is susceptible to numerous disorders, diseases and optical deficiencies. Corrective glasses, contact lenses or laser sculpting typically addresses optical deficiencies. Besides optical deficiencies, several diseases that can affect the natural crystalline lens or the optical nerve 108 or macula 109 can degrade vision. For example, cataracts interfere with vision by causing a cloudy or opaque discoloration of the natural lens 102 of the eye. Cataracts often result in partial or complete blindness. If this is the case, as shown in FIG. 2 the crystalline lens can be removed and replaced with an intraocular lens (IOL) 16. Cataract lens removal in a myopic patient, particularly a high myopic, presents addition optical problems and may result in retinal detachment and macular degeneration.

Intraocular lenses (IOLs) 16 have proven to be very successful in restoring normal vision to individuals following removal of a natural crystalline lens 102 clouded by the presence of a cataract. While there are many different designs of IOLs and various different placement procedures, methods for positioning and retaining the IOL in position once placed and various different locations for placement of the lens, a typical state of the art IOL 122 is placed within the lens capsule 104 (which is a membrane bag enclosing the natural lens 102) after removal of the clouded natural lens. While posterior chamber IOLs 122 may have different shapes and dimensions for the IOL optical portion and peripheral retaining structure, a typical posterior IOL 122 has an optic diameter of about 6 mm and an optic thickness of about 1.5 mm thick.

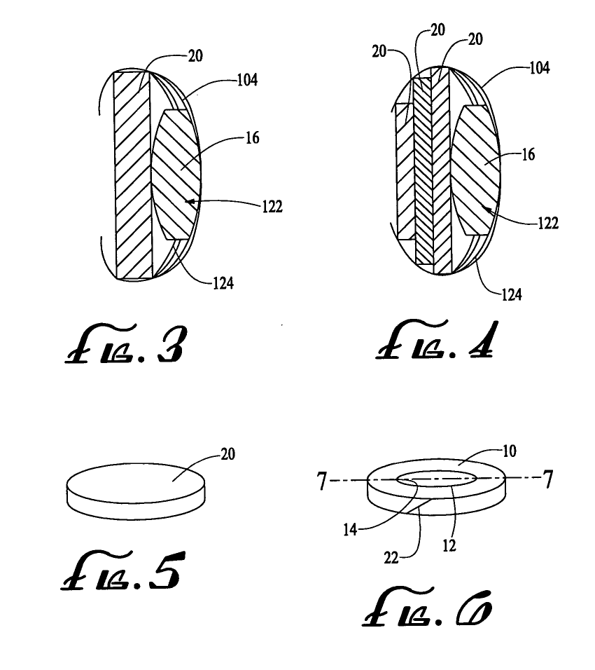

Because a typical lens capsule 104 has a mean diameter of about 10.5 mm (range 10.65-12.0 mm), the IOL also typically has haptics, flanges or other positioning structure 124 extending outward from the optical portion 16 of the IOL to keep the optical portion 16 centered within the lens capsule 104 with a central axis generally coinciding with an axis through the center of the iris. As shown in FIG. 3, the haptics, flanges or other positioning structure 124 may also be mounted at an angle to the plane of the optical portion 16 of the IOL 122 so that the optical portion 16 is pushed rearward against the remaining membrane (referred to as the posterior or hyloid membrane) 126 forming the rear of the lens capsule 104.

While the normal human natural lens 102 is thicker in its center than an IOL optic 16, this may not present a problem. However, in some individuals and in particular individuals considered to be a high myopic (requiring an optical correction greater than about 10 diopters) the natural lens 102 may have a thickness as great as 5 mm. Following implantation of an IOL 122 the posterior capsule 104 tends to shrink and wrap around the IOL 122. Because of the greater thickness of the removed lens 102, the shrinking capsule can result in the loss of several millimeters of capsule 104 depth, the remaining posterior capsule 104 shifts forward, aqueous fluid 112 flows from the anterior chamber 114 through the iris 110 filling the remaining volume of the capsule 104 and vitreous gel 116 posterior of the capsule membrane 126 may move forward.

The arrows within the eye structure shown in FIG. 2 represent the forward movement of the capsule and the vitreous. This forward movement of the vitreous 116 can cause a retinal detachment 128 and initiate macular degeneration. However, IOLs 122 have not been found to adequately prevent the forward movement of the rear wall of the capsule.

Another problem that can occur is that the refractive outcome of the IOL procedure is not predictable if the exact location (i.e., position of the IOL in relationship to the cornea and retina) is not known. The effective power of the implanted IOL changes as a function of its position within the eye. If the position is not closely controlled, the refractive outcome will be different from that desired. In order to maintain the spacing in the eye, or modify that spacing, and to select the proper dimensioned IOL and spacing means, it is important not only that the dimensions of the eye structures and the spatial relationship between these structures be known preoperatively but that those dimensions and spatial relationship be maintained.

While the method described herein is applicable to various different ophthalmic surgical procedures which may result in a change in the spacing of the structures within the eye, the method is described in regard to one embodiment comprising the removal of a cataract-containing crystalline lens, the replacement of that lens with an intraocular lens and insertion of spacing devices to position the posterior surface of the capsule in substantially the same position it was preoperatively or some other medically preferred location. However, the method described herein is not limited to an IOL procedure or use of the spacers or rings described herein but is generally applicable to a broad range of ophthalmic surgical procedures which may disrupt the preoperative spatial relationships.

In order to position an IOL more rearward in the posterior capsule 104 to maintain capsule dimensions such as shown in FIGS. 3, 4 and 8 the dimension and spacing of the patients eye are determined preoperatively. Numerous instrumental techniques have been developed to measure dimensions within an eye. These include, but are not limited to ultrasound, partial coherence interferometry and optical coherence tomography which allow dimensions within the eye to be measured non-invasively. Various laser measuring techniques are also available. Oculus USA describes the PENTAGRAM® eye scanner which uses a rotating Scheimpflug camera to obtain images of the interior structure of the eye. This system also provides numerical values for the various positional relationships of these structures, particularly the anterior and posterior segment of the eye (http://www.oculususa.com/prd comp.php). The method also contemplates generating digital images of the eye's internal structure preoperatively and substantially duplicating the spatial relationships in that image postoperatively by placing spacing means in the eye as part of the surgical procedure, thus establishing a desired postoperative structural spatial relationships.

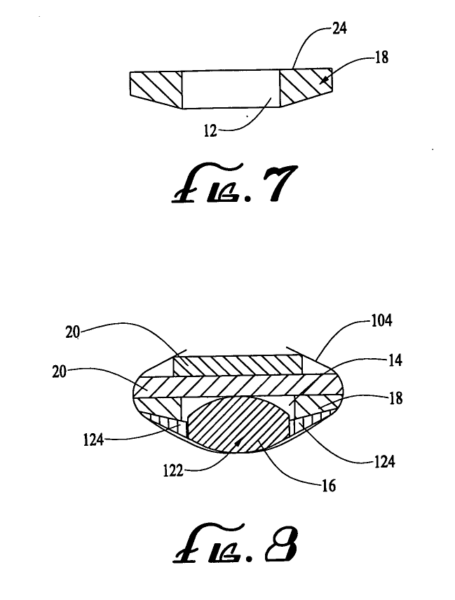

While techniques to measure the dimensions of the eye are available, this information has not generally been considered to be clinically useful. The use of spacing means, such as the spacers set forth herein, allows the surgeon to maintain the dimensions in the eye and provide surgical relevance to measuring techniques previously developed. Once the dimensions or spatial relationships are established preoperatively, the surgical procedure, such as the cataract removal procedure, can then be performed, an IOL implanted and various devices can be placed anterior to the IOL to reestablish the measured spacing. These devices can comprise one or more spacers which may be in the form of a ring 10, 18 with a central hole 12 having a diameter 14 approximating that of the IOL optic 16 as shown in FIGS. 6 and 7. The device may also be a disk 20, such as shown in FIG. 5, which covers both the optic 16 and the haptic 124 of the IOL. However, other devices such as inflatable structures, thicker lenses or multiple lenses can be placed in the eye to reestablish the original spacing, dimensions and spatial relationships between the structures in the eye. Interoperative measurement of the eye using the same or similar techniques can be performed to assure that the dimensions of the selected implants results in substantially the same dimensions and spacing within the eye, or new dimensions and spacing if desired, before the surgical incision is closed.

The art discloses numerous references to the use of laser beams for measurement of distances. A particularly useful technique, both preoperatively and intraoperatively, is to use two HeNe beams. The beams are simultaneously focused on two different structures in the eye, for example on the posterior capsule wall and on the posterior surface of the cornea, and a digital micrometer is used to measure the distance between those two points of focus. Alternatively, a single laser beam or more then two beams can be focused to either one of the focus points and then to the other of the two focus points, or simultaneously to the two surfaces or focus points, and the difference between the two points determined. Once the preoperative distance is determined, since the thickness of the various implantable components (IOL, spacers, rings, discs, etc) are known or can be measured, the proper implantable components to maintain or adjust the intraocular space can be selected preoperatively. Preoperative measurement of the various dimensions also aids in selecting the type and shape or thickness of the intraocular lens, and adding loops, rings, plates or other structures to also maintain or adjust not only the depth but also the circumference of the capsular bag.

The collapse of the enlarged capsule of a high myope following cataract removal and IOL placement in the capsule is addressed by placement of one or more transparent rings 10, 18, plates or discs 20 against the anterior face of a posterior chamber IOL 122 and/or the haptic 124 thereof. If rings 10, 18 are used, the centrally located open space 12 within the ring 10 typically has a diameter 14 approximating the diameter of the IOL optic 16, generally greater then about 6mm, the ring 10, 18 being located anterior of the IOL optic 16 and the haptic 124. Alternatively, one or more discs 20 can be stacked on the anterior surface of the IOL but still within the capsule 104.

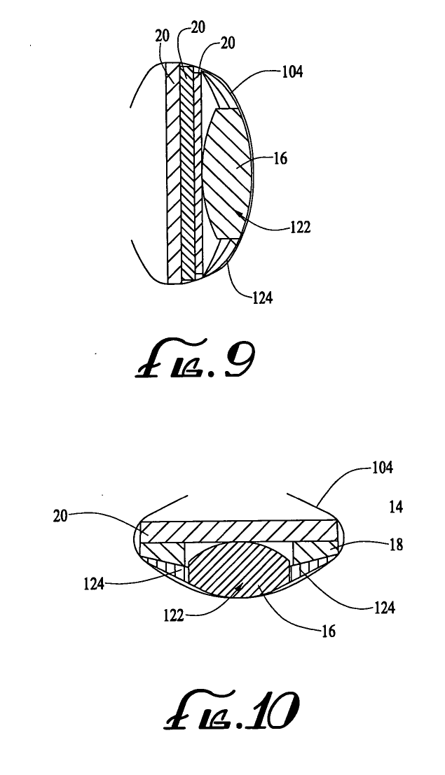

Still further, a combination of discs 20 and rings 10, 18 can be used such as shown in FIGS. 8 and 10. These discs 20 or rings 10, 18 serve as spacers and are not intended to provide any optical correction. Their primary purpose is to maintain the normal depth of the capsule 104, push the IOL optic 16 rearward so that it makes uniform contact with the posterior membrane 126 of the capsule and maintains the normal location of the capsule membrane 126 so that the vitreous 116 does not move forward, which can cause retinal detachment and start macular degeneration. The ring 10, 18 or disk 20 can also have square edges to act as a barrier to prevent migration of epithelial cells along the back of the IOL 122 and thus prevent or retard posterior capsular opacification.

Since the discs and/or rings push the IOL rearward away from the typical central position for an IOL, and the location of the IOL can be determined preoperatively the optical correction provided by the IOL can be selected to compensate for the defined position and to provide for the proper optical correction for the patient.

FIG. 5 is a schematic drawing showing an embodiment of a disc 20 incorporating features of the invention. FIG. 6 is a schematic drawing showing a first embodiment of a ring 10 having parallel faces. The spacers, whether in the form of a disc 20, or ring 10, 18, may be constructed of numerous biomaterials typically used for ophthalmic implants including, but not limited to, rigid biocompatible materials such as polymethyl methacrylate (PMMA) or polycarbonate or, preferably, deformable materials such as silicone, acrylic or hydrogel polymeric materials, and the like. If a single spacer (disc 20 or ring 10, 18) is used, a typical spacer would have an outer diameter to match the normal diameter of the patients capsular bag 104 when enclosing a natural lens 102, typically 9.5-13 mm, and a thickness of from about 0.5 to about 3 mm. If multiple rings 10, 18 or discs 20 are used the thickness of the largest diameter spacer, which is preferably the spacer contacting the anterior surface of the IOL 122, can be reduced to compensate for the thickness of the other spacers. The other spacers would typically have smaller diameters so that they form a truncated pyramid when stacked on top of each other without spaces there between, such as shown in FIGS. 4 and 8. While shown in the figures as a planer disc, when implanted the planer spacer contacting the IOL would preferably contour to the surface of the IOL. This would occur without effecting the optical correction provided by the IOL. A typical single ring 10, 18 or disc 20 has an outer diameter of about 11-12 mm and a thickness from about 0.5 mm to about 3 mm, but may be thicker if the posterior chamber is unusually deep. If multiple discs 20 or rings 10, 18 are used they can have different thicknesses but the total thickness of a stack of discs or rings would chosen to reproduce, in combination with the IOL thickness, the normal depth of the capsular chamber in the patient. Alternatively, as shown in FIG. 9, the spacers can be stacked with the smallest diameter disc against the 10L or the largest disc may be between smaller diameter discs. In other words, the various diameter spacers can be stacked so the outer contour of the stack matches the natural internal contour of the capsule.

When rings are used, it is preferred that the central hole 12 there through is equal to or greater than the diameter of the IOL optic 16 so that the inner edge of the ring 10, 18 is not within the optical path of an image being observed through the IOL 122 as this can distort the image and cause glare. Also, because the ring 10 does not directly push on the optic 16 but instead moves the optic 16 rearward by pressing on the haptic 124, the ring 18, as best shown in FIG. 7, can also have an angled rear surface, for example matching the angle of the haptic 124 from the plane of the optic 16. FIG. 7 shows a ring 18 with an angle to match that of an angled haptic 124, typically about 6°.

On the other hand, discs 20 preferably have parallel front and rear surfaces so that they do not add to or subtract from the optical characteristics of the IOL 122. While they press against the IOL optic 16, which may cause the optic 16 to be spaced rearward, the primary intended finction is to also space the haptic 124 rearward, causing rearward movement of the optic 16. For this reason, there is value in combining a ring 10, 18 with a disc 20 and particularly the ring 18 with angled rear surface such as shown in FIGS. 7, 8 and 10.

While multiple discs are shown, the same finction can be obtained by use of a single disc and/or ring combination that duplicates the configuration of the multiple discs. For example, while FIGS. 4 and 9 show three stacked discs, a single disc can provide this configuration with a stepped edge or a contoured edge that approximates the contour of the capsule in which it is to be placed. In the same manner, the ring and two discs shown in FIGS. 8 or 10 can be provided as a single spacer having the same or similar outer contour and a posterior opening to receive the optic of the IOL.

As a further variation, the disc 20 or ring 10, 18 does not have to be a solid material. It can be an inflatable disc 20 or ring 10, 18 that can be filled with a liquid before or after placement to create the desired spacer dimensions. This can also provide an opportunity to vary the dimensions of the spacer once implanted by adding or removing the filler material.

As a still further variation, if the disc 20 or ring 10, 18 is flexible, it may also provide accommodation if the zonules in the eye are still intact, causing the IOL optic 16 to move in response to the eye trying to focus on images at different distances. While the rings and disc are shown as solid structures, the same spacing effect can be obtained by proving a notch in the disc or a slot across the ring providing an opportunity for the disk to contour more readily to the IOL or the ring diameter to be increased or decreased after implantation. In such instance, to prevent cell migration through the slot of notch it is preferred to use two or more of the spacers with the notch or slots oriented in different directions to present a tortuous movement path for the migrating cells. As a still further alternative, the ring can be provided with an angled cut 22 through the toroidal portion 24 of the ring so that the surfaces along the angled cut can be displaced as the diameter of the ring is increased or decreased. This allows for slight variation in the ring diameter without providing access for cell migration.

While the method has specific benefit for use in myopic patients who may have a lens capsule with a greater depth then normal, it is also contemplated that the method shown and described herein can be used in patients with normal capsule dimensions because IOL lenses are usually of a lesser depth (thickness) than the natural crystalline lens which the IOL replaces. The described procedure provides for proper selection of the IOL with the desired refractive properties and then placement of the IOL within the eye at the optimum desired location for proper corrected vision. Also, while the method described has particular utility in cataract procedures, it also can be used for patients who require a lens removal and replacement for purposes other then replacement of a crystalline lens clouded by cataracts as well as for other surgical procedures. In addition, the method is not limited to the specific spacers or rings described herein. Many different devices can be implanted in the eye, including but not limited to inflatable structure and thick or multiple lens or physiologically acceptable liquids which remain where placed in the eye and are not dissipated over a period of time.

The method contemplates that devices or materials placed in the eye as part of the surgical procedure allow the surgeon to determine the preoperative spacing and spatial relationships of structures in the eye, perform the surgical procedure and as part of the procedure implant devices or materials to duplicate or adjust, immediately post operatively, the preoperatively determined spacing of the anterior and/or posterior capsule. While it is desirable in most procedures to substantially duplicate the preoperative relationships postoperatively, the method also contemplates establishing a different but predetermined spatial relationship between the structures as may be required by the intended end result of the surgical procedure. In such an instance preoperative measurements are made, the surgical procedure is performed and spacing means are placed within the eye to obtain the intended adjustment of intraocular spaces. One skilled in the art will recognize that, based on the disclosure herein, variations on the method can be made without varying from the invention disclosed herein and the invention is limited only by the claims set forth below.

1. A method of maintaining normal spatial relationships for a particular patient, or modifying in a controlled manner the spatial relationship, between structures within the eye in a ophthalmic surgical procedure comprising preoperatively determining the spatial relationship between structures within the eye, performing the ophthalmic surgical procedure, placing spacing means within the eye and closing the surgical incision, such that the spacing means placed within the eye provides a predetermined spatial relationship between said structures within the eye postoperatively.

2. The method of claim 1 wherein the surgical procedure is a cataract removal procedure, the depth of a posterior charnber of the eye is predetermined preoperatively by determining the spatial relationship between the posterior lens capsule and the cornea, selecting one or more structures for implantation in the lens capsule following removal of a natural crystalline lens, the thickness of the one or more structures producing a desired spacing of the lens capsule from the cornea and the retina.

3. The method of claim 2 wherein the desired spacing approximates the spatial relationship of the lens capsule measured preoperatively.

4. The method of claim 2 wherein the desired spacing is a predetermined difference from the spatial relationship of the lens capsule measured preoperatively.

5. The method of claim 2 wherein the one or more structures for implantation in the lens capsule comprise one or more of an intraocular lens and spacing means.

6. A method of maintaining the normal depth of a posterior chamber of the eye in a procedure for removing a lens from a lens capsule comprising preoperatively measuring the distance between the posterior of the lens capsule and the cornea, or the posterior of the lens capsule and the retina or the cornea and the retina, selecting an intraocular lens and one or more spacing means for implantation in the lens capsule following removal of a natural crystalline lens, the thickness of the one or more spacing means in combination with the intraocular lens producing a depth for the lens capsule approximating the depth of the lens capsule measured preoperatively.

7. The method of claim 6 further including measurement of the distance between the posterior lens capsule and the cornea, or the posterior of the lens capsule and the retina or the cornea and the retina, after placement of the intraocular lens and the one or more spacing means to verify the dimensions are substantially as intended.

8. The method of claim 6 wherein the measurement of the distance between the posterior lens capsule and the cornea, or the posterior of the lens capsule and the retina or the cornea and the retina, is determined using one or more lasers focused on locations on one or more of the posterior lens capsule, the cornea and the retina and instrumentation to determine the distance between said focus locations.

9. The method of claim 5 wherein the measurement of the distance between the posterior lens capsule and the cornea, or the posterior of the lens capsule and the retina or the cornea and the retina, is determined using ultrasound, partial coherence interferometry, optical coherence tomography or optical, photographic or digital imaging.

10. A method of maintaining the normal depth of a posterior chamber of the eye and preventing forward movement of the vitreous and retinal detachment following removal of a crystalline lens and placement of a posterior chamber intraocular lens in the posterior chamber of the eye comprising preoperatively determining the distance between the posterior lens capsule and the cornea, or the posterior of the lens capsule and the retina or the cornea and the retina, selecting one or more spacing means and an intraocular lens such that the thickness following implantation of a combination thereof within the posterior chamber establishes a predetermined distance and placing the intraocular lens and one or more spacing means in the posterior chamber.
